(19) United States
(12) Patent Application Publication
(10) Pub. No.: US 2005/0106642 A1
(43) Pub. Date: May 19, 2005

(54) Saliva test for early diagnosis of cancers

(76) Inventors: Binie V. Lipps, Bellaire, TX (US); Frederick W. Lipps, Bellaire, TX (US)

Correspondence Address: John R. Casperson, PO Box 2174, Friendswood, TX, 77549, US

(21) Appl. No.: 10/716,982

(22) Filed: Nov. 19, 2003

(51) Int. Cl. G01N 33/574; C12P 21/04

(52) U.S. Cl. 435/7.23; 435/70.21

(57) ABSTRACT

Proteonic cancer markers (PCMs) for breast, colon, liver and ovary were isolated, from the respective lysate of transformed cells, by differential centrifugation. Polyclonal antibodies were generated in mice against the (PCMs) for breast, colon, liver and ovary individually and combination thereof. Saliva from normal people was assayed by ELISA for antimixture of PCMs; breast, colon, liver and ovary cells individually. It was revealed that cancer antigen was detectable in saliva from normal people and the ELISA titer/100 μl ranged from 1:200 to 1:1600. Out of 32 normal salivas tested, ELISA titer was higher than 1:1000 in seven specimens. Those specimens were assayed by ELISA tests for individual PCM using anti-breast, anti-colon, anti-liver and anti-ovary. Each saliva specimen showed highest titer for one type of cancer antigen. Four saliva specimens showed high titers for breast PCM, two for colon one for liver. Only one saliva specimen showed high titer for ovary and colon PCMs. Thus, the invention further relates to the quantitative assessment of specific PCMs for breast, colon, liver and ovary in human saliva, by using antibodies against these markers individually.

### BACKGROUND OF THE INVENTION

This invention relates generally for screening and diagnosing of early cancers by using noninvasive saliva test.

Cancer is a general term for the abnormal growth of cells. In prosperous countries, roughly 20% or one in five people will die of cancer. The most frequently occurring cancers worldwide in descending order are stomach, lung, breast, colon/rectum, cervix and mouth/pharynx.

Early diagnosis of cancer is critical for monitoring successful treatment. Late diagnosis increases the risk that the cancer has metastasized and severely limits treatment options. A procedure to facilitate early diagnosis would be desirable.

Bodily fluids in cancer victims are known to contain chemicals called tumor markers. Breast, lung, and bowel tumors, for example, produce a protein called the carcinoembryonic antigen (CEA). If a very high CEA level is found, then a tumor is assumed to be present. A low level of CEA may be associated with inflammatory diseases. Similarly, prostate cancers produce prostrate cancer antigens and many cancers of the testicles and ovaries are known to produce chemicals. However, the existing tests are not very specific or reliable and therefore have not found wide use. A specific and reliable test to screen for cancer would be very desirable.

Another advantage of a specific and reliable test would be its use to monitor the effectiveness of treatment. If the marker is elevated at the time of diagnosis, then successful treatment should result in the level falling or disappearance altogether. The reappearance of the marker would signal a relapse. Again, however, the existing tests are not in widespread practice because sometimes they give false negative results.

Studies have shown that screening of women aged 50 and over by mammography followed by treatment reduced the mortality by 20% to 40%. However, such reduction was only 13% to 20% in younger women. In addition mammography tends to give false positive and false negative diagnoses for breast cancer. These results demonstrate that a better and more specific screening test for the diagnosis of breast cancer is needed. The same can be said for the prostate cancer diagnostic test, which is not specific and gives false positive and negative results. Therefore, none of these tests are popular because the diagnosis of cancer is not reliable.

Much work has been done in identifying tumor markers in blood and tissues. However, these substances are very complex and samples cannot be obtained without invasive procedure. For example, blood is a complex mixture of many different proteins, and blood serum may become milky when lipemic, or red when red blood cells are hemolyzed due to the liver disease. The presence of many proteins complicates assaying for trace amounts of tumor markers and may result in a greater risk of nonspecific interference and a greater chance for hydrostatic (and other) interactions between the factors of interest and other serum proteins. The color variations in normal and disease altered serum can also affect colorimetric assays such as ELISA, making it difficult to produce a consistent blank and interfering with the true values of the serum assay when compared to the consistent clarity of the assay standards. Further, blood serum analysis requires a double sandwich ELISA protocol and the collection procedure itself is invasive and not without risks. A procedure in which a simple-to-analyze specimen can be collected non-invasively would be very desirable.

Streckfus et al., U.S. Pat. No. 6,294,349, issued Sep. 25, 2001, discloses the identification of erb and CA 15-3 breast cancer markers in human saliva as well as tumor suppressor oncogene p53. The identification was carried out by using kits from three different companies, Triton Diagnostic, CIS bio-international and Oncogene Research Co. to assay for the markers. Cancer antigen 15-3 was indicated to be at least about 100% higher in saliva of women when a malignant tumor was present than in the controls. Tumor suppressor oncogene p53 was indicated to be at least about 25% lower in subjects having malignant breast tumors than the controls. Chen et al. (Chen Di-Xia et al. “Saliva and serum CA 125 assays for detecting malignant ovarian tumors”, PG, vol. 75, (1990)) found that saliva contained CA 125, a glycoprotein complex, that recognized specific tumor markers for ovarian cancer. It is reported that immunoglobulins IgG and IgA were assayed from saliva of Balb/c mice. It is advantageous to use bodily fluids such as saliva, tears and sweat collected non-invasively.

### OBJECTS OF THE INVENTION

There is a need for a reliable test for screening and for diagnosing early cancers. It is an object of the present invention is to provide a novel saliva test to diagnose early cancer, based upon the concentration of cancer markers in the saliva. A further object of the invention is to detect and quantify cancer markers in saliva by simple ELISA test. Another object is to identify cancer markers in saliva specific to cancers of the breast, colon, liver and ovary to facilitate proper treatment and therapy. Another object of the invention is to provide a test which can be used to monitor the efficacy of drug treatment for cancer. High levels of cancer antigens before chemotherapy, then successful treatment, should result in the level falling down. The reappearance of cancer markers would signal a relapse. Under such condition saliva should be retested in search of other types of cancer markers also.

### SUMMARY OF THE INVENTION

We have isolated specific proteonic cancer markers (PCMs) from their respective cancer cells, such as human breast, colon, liver and ovary cancers.

We have made polyclonal antibodies in mice against the isolated PCMs individually and against mixtures of PCMs.

We have perfected a simple ELISA test to assay for the presence of PCMs from saliva.

The test is useful for the early diagnosis of cancer in general and cancers of the breast, colon, liver and ovary specifically from saliva samples.

The novel saliva test will make possible (1) Diagnosis of specific type of cancers, for example, cancers of the breast, colon, liver and ovary. The diagnosis of other types of cancers; for example, lung, stomach, pancrease etc. can be achieved by using PCMs specific for these cancers (2) The diagnosis of cancer type is accomplished by this novel test, will promote proper chemotherapy. Furthermore, (3) the test can be used to monitor the efficacy of treatment, chemotherapy and radiation. Successful treatment should lower the amount of PCMs present. Currently, computerized tomography (CT) scan is the only available test for determination of the size of the tumor, before and after chemotherapy. (4) This novel, more specific test, can minimize the use of CT scan and reduce X-ray exposure caused by CT scan. (5) The saliva test will never be falsely negative. Because, we all carry genes for cancer and at any given time mutated cells are bound to be present, so also the proteonic cancer markers. It is the level of the concentration of marker that identifies the precancerous and cancerous state.

A first embodiment of the invention provides a non-invasive cancer screening method. A saliva specimen is obtained from the normal population not diagnosed for cancer to be screened and is formed into a saliva sample. The saliva sample is then brought together with a reagent containing antibodies made against a mixture of plurality of proteonic cancer markers from different types of cancer cells to form an assay sample. A determination is then made as to whether an immunological reaction has occurred in the assay sample. The occurrence of the immunological reaction is indicative of cancer in the human from which the saliva sample was obtained, especially when the occurrence of reaction is confirmed by ELISA test above some predetermined value.

In a second embodiment of the invention, reagents containing antibodies made against individual proteonic cancer markers are brought together with a multiple parts of human saliva sample to form assay samples. The determination is then made as to whether an immunological reaction has occurred in each assay sample. The highest occurrence of the immunological reaction to the individual proteonic cancer marker is indicative of specific type of cancer in the human from which the sample was obtained. For example, if the reaction is highest for breast proteonic cancer marker, then it is breast cancer.

In another embodiment of the invention, there is provided a method for monitoring the effectiveness of cancer treatment regimen after the patient is diagnosed for known type of cancer, say for colon cancer. A first saliva specimen is obtained from the patient and formed into a first saliva specimen. The first saliva sample is brought together with a reagent containing antibodies made against colon proteonic cancer marker. A simple ELISA test is conducted on the first assay sample to obtain a first ELISA test result on the first assay sample. The first ELISA test result provides a baseline measurement. The patient is then treated for the cancer represented by the cancer cell line used to make the proteonic cancer marker, and, after a period of time of at least one week, the procedure is repeated to obtain a second ELISA test result which can be compared to the first to determine the effectiveness of the cancer treatment. Effective treatment will lower in this case, the concentration of proteonic cancer marker for colon.

### DETAILED DESCRIPTION OF THE INVENTION

In a preferred embodiment, a colony of cancer cells is provided and the at least one proteonic cancer marker is extracted from the colony and antibodies are formed against it. The colony of cancer cells can be formed from a publicly available cancer cell line, of which there are many. For example, the cell line can be selected from the group consisting of a breast cancer cell line, a lung cancer cell line, a stomach cancer cell line, a liver cancer cell line, a colon cancer cell line, an ovarian cancer cell line, a cervical cancer cell line, a mouth/pharynx cancer cell line, a skin cancer cell line, a pancreatic cancer cell line, a testes cancer cell line, a brain tumor cell line, and a prostate cancer cell line.

Generally speaking, the antibodies which are used in the invention are polyclonal antibodies which are formed in animals. The animals are immunized with the proteonic cancer markers to generate polyclonal antibodies. The blood containing the polyclonal antibodies is collected from the animals and further separated into a serum containing the polyclonal antibodies from the blood. The reagent is formed from the serum.

The human saliva sample is generally formed by collecting and centrifuging a human saliva specimen to separate out cells and mucin. The supernatant is then collected to form the human saliva sample.

The proteonic cancer markers are generally formed by combining at least a portion of the colony of cells with a carrier fluid, agitating the carrier fluid to disrupt the cells and form a suspension, centrifuging the suspension to separate out cell debris and nuclei, and then collecting the supernatant fluid which contains proteonic cancer marker from the colony. Preferably, the centrifugation is carried out in two stages. In first stage to separate out the cell debris and nuclei in the second stage. A portion of the supernatant fluid is then injected into the animals to be used to form the polyclonal antibodies.

For screening tests, it is preferred that the reagent contains antibodies made against a plurality of proteonic cancer markers. This can be accomplished, for example, by immunizing animals with a mixture obtained by combining different proteonic cancer markers.

A second embodiment of the invention provides a non-invasive cancer screening method. A saliva specimen is obtained from the patient to be screened and is formed into a saliva sample. The saliva sample is then brought together with a reagent containing antibodies made against a plurality of proteonic cancer markers from different types of cancer cells to form an assay sample. A determination is then made as to whether an immunological reaction has occurred in the assay sample. The occurrence of the immunological reaction is indicative of cancer in the human from which the sample was obtained, especially when the occurrence of reaction is confirmed by ELISA test above some predetermined value.

Preferably, the step of determining is carried out by simple ELISA test to obtain ELISA test results which are most preferably either titer or binding affinity. Positive results from either of these tests are indicative of the occurrence of an immunological reaction in the assay sample, and obtaining ELISA test results above a predetermined value are indicative of a screening test positive for cancer.

In such case, in accord with a further embodiment of the invention, a diagnostic method is conducted. A second saliva sample from the patient is divided into a plurality of portions, and these portions are brought together with a plurality of second reagents, a single reagent being brought together with each portion. Each second reagent contains a separate slate of antibodies made against proteonic cancer markers from different types of cancer cells, one type of cancer cells being used to form each slate of antibodies. A plurality of second assay samples is thus formed. A simple ELISA test is then conducted on each of the plurality of assay samples to obtain an ELISA test result on each, and the sample giving the most highly positive test result is then identified. The most highly positive test result is then associated with the type of cancer cells used to produce the antibodies yielding such results, thereby providing the diagnosis.

In a further embodiment of the invention, there is provided a method for monitoring the effectiveness of cancer treatment regimen. A first saliva specimen is obtained from the patient and formed into a first saliva specimen. The first saliva sample is brought together with a reagent containing antibodies made against the plurality mixture of proteonic cancer markers made from a single cancer cell line to form a first assay sample. A simple ELISA test is conducted on the first assay sample to obtain a first ELISA test result on the first assay sample. The first ELISA test result provides a baseline measurement. The patient is then treated for the cancer represented by the cancer cell line used to make the proteonic cancer marker, and, after a period of time of at least one week, the procedure is repeated to obtain a second ELISA test result which can be compared to the first to determine the effectiveness of the cancer treatment. The ELISA test results are preferably selected from titer and binding affinity and a lower value for the second test results would be indicative of effective cancer treatment.

In certain aspects, this invention relates to the detection and isolation of proteonic cancer markers (PCMs), which are specific for different types of cancer such as breast, colon, liver and ovary. The invention further relates to the quantitative assessment of specific proteonic cancer markers for breast, colon, liver and ovary cancers in human saliva, by using antibodies against these markers individually.

Production of PCMs

The cell lines used in these studies were purchased from American Type Culture Collection (ATCC), Rockville, Md. The following cancer cell lines were used to derive proteonic cancer markers:
(1) HT-29, of breast cancer;
(2) Diji (M. D. Anderson Hospital) colon cancer;
(3) Chang liver cell CCL-13 of liver cancer, and
(4) Sk-ov-3 of ovary cancer.

Cancer cells are transformed cells and the transformation is caused due to the expression of oncogenes in the cells. The soluble product of the transformed cells is a proteonic cancer marker (PCM). Each type of cancer cell has it's own identified and not yet identified cancer markers. For example, the identified oncogenes for breast cancer are erb and CA-15-3, and there may be more which are yet not identified. Therefore, each type of cancer cell most likely has array of cancer oncogene product, and releases multiple PCMs. However, each type of product should predominately be associated with the type of cancer, such as breast, colon, liver, ovary etc. from which it was produced.

In the procedure used, each cell line was grown to confluency. The cells were rinsed once with phosphate buffered saline (PBS) and then with water. The cells were suspended into a small volume of water, sonicated and centrifuged. A mixture of all four cell lines was also made in this manner. Proteonic cancer markers from each cell type and the mixture were separated by differential centrifugation to remove the cell debris and nuclei. The first centrifugation at 500 G removed cell debris. The second centrifugation at 1000-1500 G sedimented nuclei. The supernatants were collected and the protein concentrations were measured on a spectrophotometer using a protein kit from Bio-Rad (catalog 500-0006). The protein concentration for each type of proteonic cancer marker and for the mixture of breast, colon, liver and ovary PCMs were adjusted to 1 mg/ml.

Production of Polyclonal Antibodies (anti-PCMs) in Mice versus Proteonic Cancer Markers:

The animals for this research were used in compliance with US Public Health Service Policy on humane care and use of animals. The first injection consisted of PCM antigen and Freund's complete adjuvant (FCA). The subsequent injections consisted of PCM antigen and Freund's incomplete adjuvant (FICA). A dose of 50 μg/mouse was given in 0.2 ml volume three times ten days apart. Finally, the mice were bled from the ophthalmic vein and sera were collected by separation from clotted blood.

Collection of Human Saliva

Saliva from individuals was collected in a tube, was centrifuged at 1000 G in order to sediment cells and mucin and the supernatant was separated. Protein concentration of the saliva was measured by spectrophotometer. The protein content for saliva was adjusted to 200 μg/ml and stored frozen from which it was diluted in carbonate-bicarbonate buffer pH 9.4 to give the concentration 10 μg/ml for ELISA tests.

Enzyme-Linked Immunosorbent Assay (ELISA) for PCMs in Human Saliva

ELISA tests were performed in 96 well micro-plates. The wells of the plate were coated with saliva at 10 μg/ml concentration in carbonate-bicarbonate buffer pH 9.4, each well receiving 100 μl. After overnight incubation at room temperature the plate was washed three times with 0.05 PBS. The wells of the plate were blocked with 3% gelatin, 250 μl/well for 1/2 hour. Anti-proteonic cancer marker (Anti-PCM) diluted in 3% gelatin from 1:100 to 1:218700 was added to three wells for each dilution. A similar procedure was followed for assaying for PCMs specific for breast, colon, liver and ovary cancers. Antigen-antibody reaction was carried out at 37 C for 1.5 hours after which the plate was washed and was reacted with mouse horseradish peroxidase conjugated with IgG. The plate was incubated for 1 hour, washed three times and dihydrochloride-OPD (Sigma) was added to develop color reaction. The plates were read at 405 nm wave length and ELISA titer/100 μl was recorded. Results are shown in Table 1.

TABLE 1
ELISA titer/100 μ 1 of anti-proteonic cancer markers (PCMs)
to respective cancer markers.

| | ELISA titer/100 μl | | | | |
|---|---|---|---|---|---|
| | Anti-PCM | Anti-PCM | Anti-PCM | Anti-PCM | Anti-PCM |
| PCM | (Mix*) | (breast) | (colon) | (liver) | (ovary) |
| Mix | 51200 | 19200 | 14800 | 13000 | 15800 |
| Breast | 19200 | 12150 | 8100 | 12150 | 24300 |
| Colon | 12800 | 8100 | 12100 | 8100 | 24300 |
| Liver | 12000 | 8100 | 2750 | 24300 | 12100 |
| Ovary | 12800 | 11500 | 2700 | 4050 | 48600 |

*Mix PCM consisted of mixture of PCMs for breast, colon, liver and ovary and the anti-PCMs was formed against the mix by immunizing mice.

Results show that PCMs are highly immunogenic, producing remarkably high ELISA titers. The mixture of breast, colon, liver and ovary PCMs showed highest reactivity to anti-mix antibodies. Anti-mix also reacted individually with breast, colon, liver and ovary, having the higher titer to breast PCM and the lowest to liver PCM, 19,200 and 12,000 respectively. Anti-breast, anti-colon, anti-liver and anti-ovary PCMs all produced higher ELISA titer to their respective PCMs than to the other specific PCMs. The results emphasize that there is a cross reactivity among the PCMs from breast, colon, liver and ovary cells.

In order to determine whether PCMs could be detected in saliva and to provide an indication of normal ranges, saliva samples were obtained from normal population not diagnosed for cancer and samples from 32 individuals were tested by ELISA with anti-mixed PCM. Results are shown in Table 2.

TABLE 2
ELISA titer/100 μl for mix anti-PCM to human saliva from
normal population.

| Saliva specimen | Titer |
|---|---|
| Saliva-1 | 300 |
| Saliva-2 | 400 |
| Saliva-3 | 450 |
| Saliva-4 | 400 |
| Saliva-5 | 600 |
| Saliva-6 | 650 |
| Saliva-7 | 1600 |
| Saliva-8 | 1200 |
| Saliva--9 | 400 |
| Saliva-10 | 400 |
| Saliva-11 | 1250 |
| Saliva-12 | 350 |
| Saliva-13 | 400 |
| Saliva-14 | 800 |
| Saliva-15 | 300 |
| Saliva-16 | 200 |
| Saliva-17 | 1250 |
| Saliva-18 | 800 |
| Saliva-19 | 400 |
| Saliva-20 | 800 |
| Saliva-21 | 750 |
| Saliva-22 | 400 |
| Saliva-23 | 400 |
| Saliva-24 | 450 |
| Saliva-25 | 800 |
| Saliva-26 | 1200 |
| Saliva-27 | 350 |
| Saliva-28 | 3200 |
| Saliva-29 | 1250 |
| Saliva-30 | 1200 |
| Saliva-31 | 400 |
| Saliva-32 | 200 |

Results show that proteonic cancer markers in saliva of normal people were detectable by ELISA. ELISA titers/100 μl in the tested human saliva ranged from 1:200 to 1:1600. Saliva 16 and saliva-32 showed the lowest ELISA titers namely 1:200, which means the lowest concentrations of PCMs. Saliva 16 and saliva-32 were obtained from young boys aged five and ten years. The other saliva samples were obtained from the adult population. This proves the known finding that cancer incidence, and thus the presence of PCMs, increases exponentially by aging.

At this stage the titers above 1:1000 were considered as tentatively positive for early diagnosis of cancer. The saliva specimens above showing higher than 1:1000 ELISA titers were tested against anti-breast, anti-colon, anti-liver and anti-ovary PCMs individually. The results are shown in Table 3.

TABLE 3
ELISA titer/100 μl of anti-proteionic cancer markers (PCMs)
to respective cancer markers in saliva of people positive for cancer.

| | ELISA titer/100 μl for | | | | |
|---|---|---|---|---|---|
| | Anti- | | | Anti- | |
| Saliva | mix | Anti-breast | Anti-colon | liver | Anti-ovary |
| Saliva-A | 1200 | 1800 | 2700 | 1800 | 900 |
| Saliva-B | 1200 | 1800 | 900 | 900 | 750 |
| Saliva-C | 1600 | 2700 | 1800 | 600 | 900 |
| Saliva-D | 1150 | 4050 | 1800 | 2700 | 1800 |
| Saliva-E | 1300 | 1800 | 1800 | 2700 | 1800 |
| Saliva-F | 1250 | 900 | 1800 | 900 | 300 |
| Saliva-G | 1200 | 8100 | 8100 | 2700 | 4050 |

The results show that saliva-B, C and G showed highest reaction with anti-breast; saliva-A, F and G made highest reaction with anti-colon PCMs. Saliva-D and E reacted strongly with anti-liver and saliva-G with anti-ovary. Note that saliva-G reacted with anti-breast, anti-colon and to some extent anti-ovary PCMs, indicating the possible presence of multiple types of cancer.

In order to determine the probable levels of PCMs in people with diagnosed cases of cancer, saliva samples were obtained from five individuals and tested by ELISA using PCM antibodies. Results are shown in Table 4.

TABLE 4
ELISA titer/100 μl of saliva of people diagnosed for cancer to mix and
individual anti- proteionic cancer markers.

| | ELISA titer/100 μl for | | | | |
|---|---|---|---|---|---|
| | Anti- | | | Anti- | |
| Saliva of | mix | Anti-breast | Anti-colon | liver | Anti-ovary |
| Stomach | 1800 | 2700 | 3600 | 1200 | 900 |
| Lung | 2700 | 1800 | 2700 | 5400 | 2750 |
| Breast | 3600 | 8100 | 2700 | 2100 | 2700 |
| Breast | 2750 | 8150 | 5400 | 2700 | 8100 |
| Prostate/ | 450 | 300 | 900 | 850 | 450 |
| Vocal cord | | | | | |

Results of table 4 show that the saliva of the patient diagnosed for stomach cancer showed the highest ELISA titer to anti-colon PCM. The patient with lung cancer showed the highest titer for anti-liver PCM. The saliva of these patients should have reacted more if were tested with anti-stomach and anti-lung PCMs, respectively. For the stomach, the colon is the closest organ and for the lung, the liver can be considered as a closest organ. The saliva from the patients diagnosed for breast cancer reacted strongly with anti-breast PCM. Saliva from one breast cancer patient also reacted strongly with anti-ovary PCM.

In further tests, the patient with stomach cancer showed an ELISA titer of 1800 against anti PCMs., which dropped to 1350 six months after therapy. Likewise, the ELISA titer for colon PCM dropped from 3600 to 2700. This indicates that effective cancer therapy decreases the concentration of PCMs in general and also for PCMs associated with the specific organ affected.

Patient was diagnosed for prostate and vocal cord cancer, three years ago. After surgery he under went chemotherapy and radiation during three year period. The status of the concentration of PCMs in his saliva before the surgery is not known. However, undoubtedly the concentration should have been higher because of the cancer. In his case the treatment for cancer worked bringing the concentration of proteonic cancer markers to normal state.

Summarizing, we have isolated specific proteonic cancer markers (PCMs) from transformed breast, colon, liver and ovary cells, by differential centrifugation method. We have generated polyclonal antibodies in mice against the PCMs of breast, colon, liver and ovary and combination thereof. We have found that each antibody is specific to the respective PCM, giving the highest ELISA titer and that there is cross reactivity for other PCMs. We used human saliva to assay for the presence of proteonic cancer markers for breast, colon, liver and ovary in humans. We have demonstrated that using saliva is advantageous over use of blood serum, as using saliva as the test specimen permits analysis by simple ELISA, whereas using serum requires double sandwich ELISA.

The proteonic cancer markers specific to breast, colon, liver and ovary have been identified in the respective cancer cells. These markers are specific to breast, colon, liver and ovary cancers and were isolated by differential centrifugation. Antibodies to the proteonic cancer markers specific to breast, colon, liver and ovary were raised in mice. The non-invasively collected saliva can be used in place of blood serum to assay for these and presumably other proteonic cancer markers. The use of saliva enables performance of a simple ELISA test versus the complicated double sandwich test if serum is used. The novel test gives diagnosis for a specific type of cancer: breast, colon, liver and ovary by using specific anti-serum. For example anti-proteonic cancer marker for breast reveals the breast cancer. Diagnosis of cancer of other types can be accomplished by incorporating specific PCMs from those types cancer cells; such as lung, stomach, pancrease, prostate, testes etc. The novel test will be useful to promote proper chemotherapy, based upon the identified type of cancer. This novel test will never be falsely negative, and it can further be used to monitor the effect of therapy. The successful treatment should show decease in lowering proteonic cancer marker. This novel more specific test will minimize computerized tomography (CT) scan, and should further reduce the need of X-ray exposures. Early diagnosis of cancer can thereby be achieved before the tumor formation.

While certain preferred embodiments of the invention have described herein, the invention is not to be construed as being so limited, except to the extent that such limitations are found in the claims.

1. A process comprising
a) bringing together a reagent containing antibodies made against a mixture of proteonic cancer markers with a human saliva sample to form an assay sample, and
b) determining whether an immunological reaction has occurred in the assay sample.

2. A process as in claim 1 further wherein
an ELISA test is conducted on the assay sample and ELISA test results are produced to determine whether an immunological reaction has occurred in the assay sample.

3. A process as in claim 2 wherein the ELISA test results are selected from titer and binding affinity and positive results are indicative of the occurrence of an immunological reaction in the assay sample.

4. A process as in claim 1 further comprising
a) providing a colony of cancer cells,
b) extracting at least one proteonic cancer marker from said colony;
c) forming antibodies against said at least one proteonic cancer marker; and
d) forming the reagent from said antibodies.

5. A process as in claim 4 wherein
the colony of cancer cells is formed from a publicly available cancer cell line.

6. A process as in claim 5 wherein the cell line is selected from the group consisting of a breast cancer cell line, a liver cancer cell line, a colon cancer cell line, and an ovarian cancer cell line.

7. A process as in claim 4 wherein the antibodies are polyclonal antibodies.

8. A process as in claim 7 wherein the polyclonal antibodies are produced in animals.

9. A process as in claim 8 further comprising separating blood containing the polyclonal antibodies from the animals and separating serum containing the polyclonal antibodies therefrom.

10. A process as in claim 9 further comprising forming the reagent from the serum.

11. A process as in claim 1 further comprising centrifuging a human saliva specimen to separate out cells and mucin and collecting the supernatant to form the human saliva sample.

12. A process as in claim 11 further comprising collecting the human saliva specimen.

13. A process as in claim 4 further comprising
combining at least a portion of the colony of cells with a carrier fluid,
agitating the carrier fluid to disrupt the cells and form a suspension,
centrifuging the suspension to separate out cell debris and nuclei and
collecting the supernatant to complete the extracting of the at least one proteonic cancer marker from the colony.

14. A process as in claim 13 further comprising
conducting the centrifuging step in two stages, to separate out cell debris in the first stage and nuclei in the second stage, and
introducing a portion of the supernatant into the animals to be used to form the polyclonal antibodies.

15. A process as in claim 1 wherein the reagent contains antibodies made against a plurality of proteonic cancer markers.

16. A non-invasive cancer screening method comprising
a) obtaining a saliva specimen from a patient,
b) forming a saliva sample from the saliva specimen,
c) bringing the saliva sample together with a reagent containing antibodies made against a plurality of proteonic cancer markers from different types of cancer cells to form an assay sample; and
d) determining whether an immunological reaction has occurred in the assay sample.

17. A method as in claim 16 wherein the step of determining is carried out by simple ELISA test to obtain ELISA test results.

18. A method as in claim 17 wherein the ELISA test results are selected from titer and binding affinity and positive results are indicative of the occurrence of an immunological reaction in the assay sample.

19. A method as in claim 18 wherein obtaining ELISA test results above a predetermined value are indicative of a positive screening test for cancer.

20. A method as in claim 19 further comprising, in a case where the ELISA test results are above the predetermined value,
a) obtaining a second saliva specimen from the patient,
b) forming a second saliva sample from the second saliva specimen,
c) separating the second saliva sample into a plurality of portions,
d) bringing the portions of the second saliva sample together with a plurality of second reagents, a single reagent being brought together with each portion, each reagent containing a separate slate of antibodies made against proteonic cancer markers from different types of cancer cells, one type of cancer cells being used to form each slate of antibodies, to form a plurality of assay samples;
e) conducting a simple ELISA test on each of the plurality of assay samples to obtain an ELISA test result on each of the plurality of assay samples,
f) identifying a most highly positive test result, and
g) associating the most highly positive test result with the type of cancer cells used to produce the antibodies yielding such results.

21. A cancer diagnostic method comprising
a) obtaining a saliva specimen from a patient,
b) forming a saliva sample from the saliva specimen,
c) separating the saliva sample into a plurality of portions,
d) bringing the portions of the saliva sample together with a plurality of reagents, a single reagent being brought together with each portion, each reagent containing a separate slate of antibodies made against proteonic cancer markers from different types of cancer cells, one type of cancer cells being used to form each slate of antibodies, to form a plurality of assay samples;
e) conducting a simple ELISA test on each of the plurality of assay samples to obtain an ELISA test result on each of the plurality of assay samples,
f) identifying a most highly positive test result, and
g) associating the most highly positive test result with the type of cancer cells used to produce the antibodies yielding such results to provide the diagnosis.

22. A method for monitoring effectiveness of cancer treatment, said method comprising
a) obtaining a first saliva specimen from a patient,
b) forming a first saliva sample from the first saliva specimen,
c) bringing the first saliva sample together with a reagent containing antibodies made against at least one proteonic cancer marker made from a single cancer cell line to form a first assay sample,
e) conducting a simple ELISA test on the first assay sample to obtain a first ELISA test result on the first assay sample,
f) treating the patient for a cancer represented by the cancer cell line used to make the proteonic cancer marker, and, after a period of time of at least one week,
g) obtaining a second saliva specimen from the patient,
h) forming a second saliva sample from the second saliva specimen,
i) bringing the second saliva sample together with the reagent to form a second assay sample,
j) conducting a simple ELISA test on the second assay sample to obtain a second ELISA test result on the second assay sample, and
k) comparing the second ELISA test result with the first ELISA test result to determine the effectiveness of the cancer treatment.

23. A method as in claim 22 wherein the ELISA test results are selected from titer and binding affinity and a lower value for the second test results is indicative of effective cancer treatment.
